(19) United States
(12) Patent Application Publication
(10) Pub. No.: US 2026/0069615 A1
(43) Pub. Date: Mar. 12, 2026

(54) GUMMY PRODUCT PRODUCTION METHODS AND DEVICES

(71) Applicant: David Weiner, Sherman Oaks, CA (US)

(72) Inventor: David Weiner, Sherman Oaks, CA (US)

(21) Appl. No.: 19/322,687

(22) Filed: Sep. 9, 2025

Related U.S. Application Data
(60) Provisional application No. 63/692,707, filed on Sep. 9, 2024.

(51) Int. Cl. A61K 31/00; A23G 3/34; A23G 3/36; A23G 3/42; A23G 3/44; A23G 3/48; A23G 3/54; A61K 9/00; A61K 47/36; A61K 47/42

(52) U.S. Cl. CPC A61K 31/658; A23G 3/0072; A23G 3/0078; A23G 3/0085; A23G 3/368; A23G 3/42; A23G 3/44; A23G 3/48; A23G 3/54; A61K 9/0056; A61K 47/36; A61K 47/42

(57) ABSTRACT

The embodiments disclose a method including mixing a plurality of ingredients into a gelling agent ingredient to create a gummy confection mixture, heating the gummy confection mixture ingredients gradually to dissolve the gelling agent ingredient and activate its gelling properties to predetermined temperatures, fabricating a mold with a plurality of flower-shaped recesses to receive a predetermined quantity of the heated gummy confection mixture, cooling the filled mold with the heated gummy confection mixture, using sensors to monitor cooling times, temperatures, and moisture levels of the plurality of mold filled flower-shaped recesses to determine a demolding extraction time of flower-shaped gummy confections and to prevent overheating and degradation of ingredients, applying exterior coatings to the flower-shaped gummy confections after demolding extraction to enhance flavor and texture to prevent sticking and improve shelf stability, and packaging the flower-shaped gummy confections to prevent contamination.

### CROSS-REFERENCE TO RELATED APPLICATIONS

This Patent Application is a Continuation, and which claims the benefit of U.S. Provisional application entitled: “GUMMY PRODUCT PRODUCTION METHODS AND DEVICES,”U.S. Ser. No. 63/692,707 filed on Sep. 9, 2024, by David Weiner, the U.S. Patent Application being incorporated herein by reference.

### BACKGROUND

Many people have difficulty swallowing pills and capsules. This causes them to delay or not take products. In some cases, those products may be medicinal or recreational.

### BRIEF DESCRIPTION OF THE DRAWINGS

FIG. 1 shows for illustrative purposes only, an example of a gummy product silicone mold mixture fill side of one embodiment.

FIG. 2 shows for illustrative purposes only, an example of a gummy product silicone mold mixture reverse side of one embodiment.

FIG. 3A shows for illustrative purposes only, an example of a gummy product of one embodiment.

FIG. 3B shows for illustrative purposes only, an example of a gummy product dosage segment of one embodiment.

FIG. 3C shows for illustrative purposes only, an example of a gummy product dosage tear-away segment mold profile of one embodiment.

FIG. 4 shows a block diagram of an overview of a gummy product production system of one embodiment.

FIG. 5 shows for illustrative purposes only, an example of a gummy product production platform of one embodiment.

FIG. 6 shows a block diagram of an overview of gummy product ingredients of one embodiment.

FIG. 7 shows a block diagram of an overview of gummy products equipment of one embodiment.

FIG. 8 shows a block diagram of an overview flow chart of a gummy product process of one embodiment.

FIG. 9 shows for illustrative purposes only, an example of an AI nutritional gummy product recipe with daily nutritional requirements adjustment of one embodiment.

FIG. 10 shows for illustrative purposes only, an example of an AI medicinal gummy product recipe dosage adjustment of one embodiment.

### DETAILED DESCRIPTION OF THE INVENTION

In the following description, reference is made to the accompanying drawings, which form a part hereof, and which are shown by way of illustration a specific example in which the invention may be practiced. It is to be understood that other embodiments may be utilized and structural changes may be made without departing from the scope of the present invention.

It should be noted that the descriptions that follow, for example, in terms of gummy product production methods and devices, are described for illustrative purposes, and the underlying system can apply to any number and multiple types of gummy products. In one embodiment of the present invention, the gummy product production methods and devices can be configured using molds. The gummy product production methods and devices can be configured to include medicinal gummy products and can be configured to include recreational use of gummy products using the present invention.

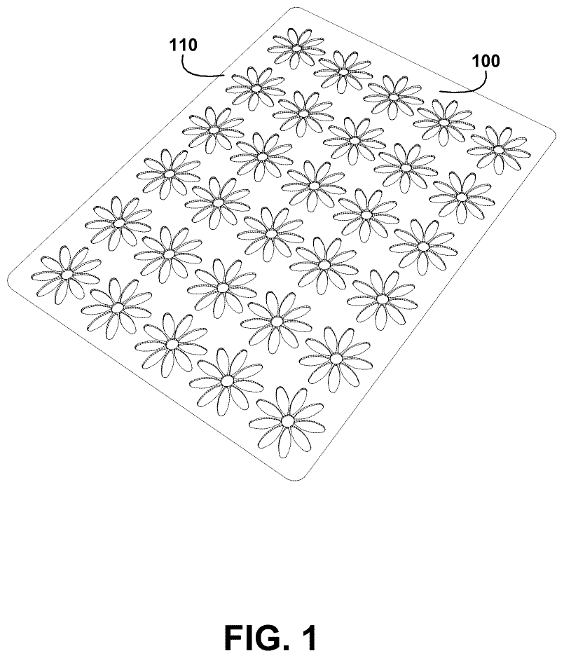

FIG. 1 shows for illustrative purposes only, an example of a gummy product silicone mold mixture fill side of one embodiment. FIG. 1 shows a silicone mold 100 shown on one side with recessed designs 110 of a gummy product. The recessed depressions are filled with a gummy mixture to process into a gummy product of one embodiment. In one embodiment, the mold 100 shown in FIG. 1 is fabricated through a casting process in which a master pattern is first generated to define the intended geometry of the gummy product. The master pattern may be produced using hand-sculpted clay, wax, or computer-aided design (CAD)-based 3D printing methods. Once the master pattern is prepared, liquid silicone rubber is poured over and around the pattern to capture fine details of the flower or other decorative designs intended for the final gummy product. The silicone is then cured to form a flexible negative mold that incorporates recessed sections 110 for receiving the gummy mixture.

After curing, the silicone mold 100 may undergo a surface preparation process to optimize performance in production. In certain embodiments, a thin layer of release agent, food-safe coating, or micro-texturing treatment is applied to the inner surfaces of the recessed sections 110. This step ensures that the gummy mixture does not adhere excessively to the mold and allows consistent demolding without damage to delicate dosage markings or tear-away features. The treatment also prolongs the operational lifespan of the mold by reducing wear and tear across repeated production cycles.

The mold fabrication process may further include a test-pour step in which a trial gummy mixture is deposited into the recesses 110 to validate dimensional accuracy, verify dosage segmentation features, and confirm cooling characteristics. Any necessary adjustments, such as altering wall thickness, depth of the tear-away lines, or curvature of the recesses, may be made at this stage before full-scale production begins. These refinements enable the mold 100 of FIG. 1 to reliably produce uniform gummies that meet regulatory standards and consumer expectations across multiple production runs.

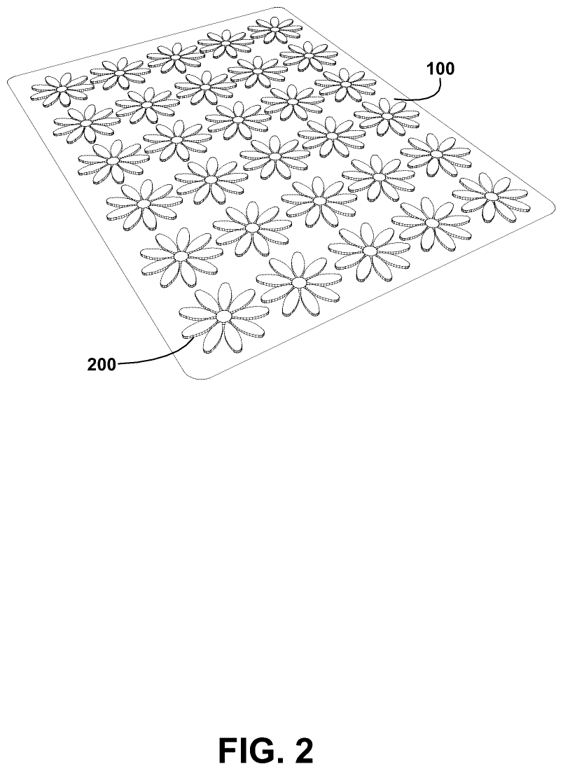

FIG. 2 shows, for illustrative purposes only, an example of the reverse side 200 of the gummy product silicone mold 100 introduced in FIG. 1. Unlike the recessed cavities on the fill side, which receive the gummy mixture during production, the reverse side 200 displays a series of raised or protruding sections that act as the inverse of those cavities. These protrusions are formed during the mold fabrication process when the liquid silicone is cured around a master pattern, creating a precise negative impression of the intended gummy product. When the gummy mixture is deposited into the recessed fill side shown in FIG. 1, the raised surfaces on the reverse side 200 serve to displace and shape the mixture, thereby producing the external contours of the final gummy product once the mixture has cooled and set.

In one embodiment, the protruding sections may include not only general shape-defining structures, such as flower petals or geometric outlines, but also finer features such as raised dosage-segmentation lines, texturing, or ornamental details. These elements are engineered into the mold so that the resulting gummy products incorporate both aesthetic characteristics and functional breakaway segments for accurate dosing. By integrating these features into the reverse side 200, the mold ensures that each gummy produced maintains uniformity, consistency in dosage, and consumer appeal. Thus, the reverse side of the silicone mold plays a complementary role to the recessed fill side by imparting the critical external details and functional design elements that distinguish the finished gummy confection.

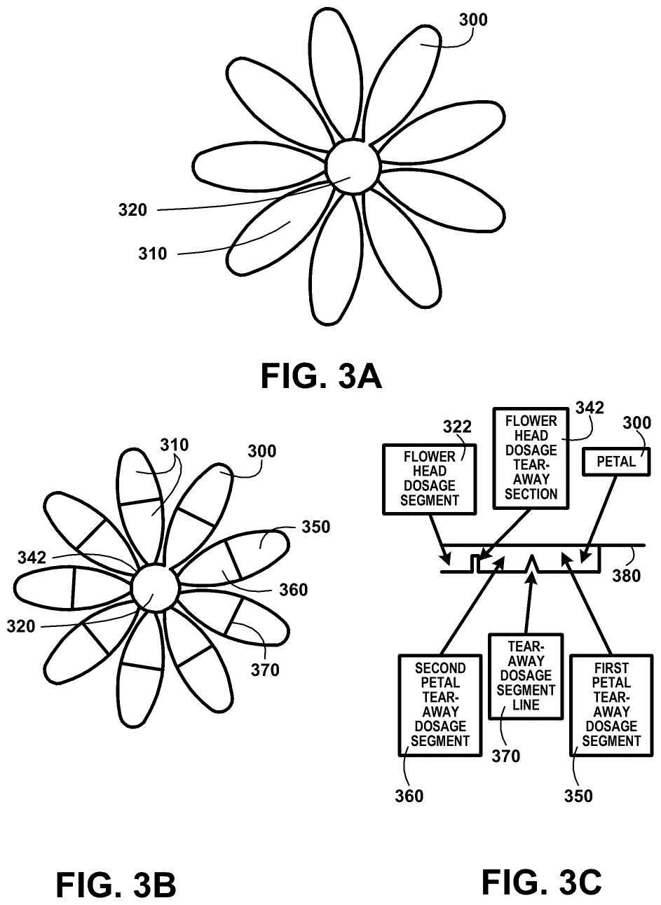

FIG. 3A shows for illustrative purposes only, an example of a gummy product of one embodiment. FIG. 3A shows a gummy product 300 produced using the silicone mold 100 of FIG. 1. The gummy product 300 is made from the gummy mixture of ingredients that is processed in a series of production steps. In one embodiment, as shown in FIG. 3A, the gummy product 300 is coated with granulated sugar 310, including the flower head 320. The coating provides a sweetener that provides a taste for the user when consuming the gummy product. In this example, the petals of the gummy product flower are separated individually for consumption of one embodiment.

FIG. 3B shows for illustrative purposes only, an example of a gummy product dosage segment of one embodiment. FIG. 3B shows the gummy product 300 is coated with granulated sugar 310, including the flower head 320. In one embodiment, the gummy product ingredients include medicinal ingredients. The medicinal ingredients can include nutritional ingredients, including, for example, vitamins, minerals, and proteins.

The medicinal ingredients can include pharmaceutical treatment ingredients, for example, cannabis derivatives including THC, CBD, and other prescription drugs in medically determined dosages for medicinal purposes. In one embodiment, the flower-shaped molded gummy is marked with lines that indicate a proper dosage amount of the drugs. The first petal tear-away dosage segment 350 and a second petal tear-away dosage segment 360 are segments with a tear-away dosage segment line 370. The segmented lines are raised line features in the mold to create tear sections for easier separation of the dosage segments, including a two-part petal with segment one and segment two 330. The flower head dosage tear-away section 342 is an additional segment with the proper dosage.

The petal 300 is connected to the flower head 320 with a narrow section 340 for easily tearing off segment two 330 from the flower head 320. The labeling provides information on the active treatment drug, proper dosages, user instructions and precautions, and other regulatory requirements. The gummy product 300 is made from a gummy mixture of ingredients that is processed in a series of production steps. In one embodiment, as shown in FIG. 3B, the gummy product 300 is coated with granulated sugar 310 for taste.

The following describes certain packaging and labeling conventions that could be used in one embodiment when medicinal THC or CBD is used for human consumption. Local and federal rules are constantly changing - the below descriptions are examples of what can be used. Labeling requirements for THC and CBD-infused gummy products are to ensure consumer safety, compliance with regulations, and clear communication of product details for medicinal purposes. While specific requirements vary depending on the state, or local jurisdiction, there are several general categories of information that are typically required on labels. Below is a description of the most common labeling requirements for THC and CBD-infused gummies for medicinal purposes.

Product Information includes a product name wherein the name should clearly indicate what the product is, such as “THC Gummies” or “CBD Gummies.” Cannabinoid content includes THC and CBD content per serving for medicinal purposes. The label should specify the amount of THC and/or CBD in each individual gummy, usually in milligrams (e.g., 10 mg of THC per gummy). Total THC and CBD content on the label should also include the total amount of THC and/or CBD in the entire package (e.g., 100 mg THC in the entire package of 10 gummies). A cannabinoid profile, for example, includes the full cannabinoid profile, listing other cannabinoids such as CBN, CBG, etc.

A full list of ingredients should be provided, similar to any food product, with both active ingredients (like THC and CBD) for medicinal purposes and inactive ingredients (like sugar, pectin, or flavorings). Allergen warnings are shown if the gummies contain or were produced in a facility that handles common allergens (e.g., nuts, soy, dairy).

Dosage Information is shown in a serving size. The label should clearly indicate the serving size, especially for products containing THC for medicinal purposes, to help consumers control their intake. Suggested use as some jurisdictions recommend including usage instructions or suggested dosing information (e.g., “Start with one gummy and wait 2 hours before increasing dosage”).

Safety warnings, for example, a THC warning where products containing THC should include a prominent warning that the product contains cannabis or THC for medicinal purposes, often using a standardized symbol (e.g., a red triangle or the letters “THC”). A warning that THC may cause intoxication, such as: “This product contains THC and may cause psychoactive effects.” Additional health and safety warnings include “Keep out of reach of children and pets.”; “Do not drive or operate heavy machinery after consuming.” and “Not for use by individuals under 21 (or 18) years of age.” Some jurisdictions require labels to mention potential side effects, such as dizziness, drowsiness, or anxiety. Some states require specific symbols or logos on cannabis products to indicate compliance with local laws. For example, California requires the universal cannabis symbol (a marijuana leaf inside a triangle).

Testing information on labels includes a statement indicating that the product has been tested by an accredited third-party laboratory, including testing for potency, contaminants, and residual solvents. The label should include a batch or lot number that can be traced back to the manufacturing process for quality control and recall purposes. The date the product was manufactured should be included, especially for products with cannabinoids that may degrade over time. Some jurisdictions require an expiration date to ensure consumers are aware of when the product may lose potency or freshness.

Packaging requirements include Child-Resistant Packaging. The product should be in packaging that is designed to be difficult for children to open, in compliance with the Poison Prevention Packaging Act (PPPA) or similar regulations in other jurisdictions. The packaging should have tamper-evident seals to ensure the consumer can see if the package has been opened before purchase.

The Certificate of Analysis (COA), while not always required on the label, it is recommended to include a QR code or link to the product's Certificate of Analysis (COA), which details the lab results for THC/CBD potency, and testing for pesticides, solvents, and microbial contaminants. Also, labels for THC and CBD products should not make unapproved health claims such as “cures cancer” or “treats anxiety.” The FDA and similar regulatory bodies prohibit these claims unless backed by substantial evidence and approval. An example of a label breakdown includes on the front of the package the Product Name, Cannabinoid content, serving size, and a universal cannabis symbol. The back of the package should include a full ingredient list, dosage information, safety warnings, testing information, batch number, manufacturing, and expiration dates, and a QR Code for COA of one embodiment.

The gummy product for use in consuming nutritional ingredients and medicinal ingredients is used in one embodiment for a user to consume than tablets and pills or poor-tasting liquids. The flavorings and tastes, including fruit flavors and chewing like many confections, allows the users to be more familiar with these gummies for more frequent consumption in one embodiment.

Both THC (tetrahydrocannabinol) and CBD (cannabidiol) are cannabinoids derived from the cannabis plant and have various medical uses. The legal status of these uses depends on jurisdiction, but in many U.S. states, THC and CBD are approved for specific medical conditions. A list of the common legal medical uses of each ingredient includes legal medical uses of THC.

In one embodiment, for medicinal purposes, the gummy product with THC or CBD can be used for chronic pain management to manage chronic pain from conditions such as arthritis, multiple sclerosis, and fibromyalgia. Also, the gummies can be used for nausea and vomiting relief (especially in chemotherapy patients) can be used. In one embodiment, for medicinal purposes, the gummy product with THC or CBD can be used for appetite stimulation (cachexia) for medicinal purposes to stimulate appetite in people with cachexia, particularly in HIV/AIDS patients and individuals undergoing cancer treatments.

In one embodiment, for medicinal purposes, the gummy product with THC or CBD can be used for muscle spasticity (multiple sclerosis) for medicinal purposes to help reduce muscle spasticity and related symptoms in people with multiple sclerosis and other neurological conditions. In one embodiment, for medicinal purposes, the gummy product with THC or CBD can be used for intraocular pressure in glaucoma patients. In one embodiment, for medicinal purposes, the gummy product with THC or CBD can be used to help manage post-traumatic stress disorder (PTSD) symptoms. In one embodiment, for medicinal purposes, the gummy product with THC or CBD can be used as a sedative that can aid in the treatment of insomnia and other sleep-related conditions for medicinal purposes.

A list of the common legal medical uses of each ingredient includes legal medical uses of CBD. In one embodiment, for medicinal purposes, the gummy product with THC or CBD can be used for medicinal purposes to manage generalized anxiety disorder, social anxiety disorder, and panic disorder. In one embodiment, for medicinal purposes, the gummy product with THC or CBD can be used for chronic pain and inflammation for medicinal purposes to alleviate chronic pain and inflammation, especially for conditions like arthritis, back pain, and fibromyalgia, without causing intoxication. In one embodiment, for medicinal purposes, the gummy product with THC or CBD can be used for medical uses for neuroprotective properties (Alzheimer's and Parkinson's) in managing neurodegenerative conditions like Alzheimer's disease and Parkinson's disease by reducing inflammation and oxidative stress in the brain.

In one embodiment, for medicinal purposes, the gummy product with THC or CBD can be used to help manage cravings and withdrawal symptoms for individuals recovering from addiction to substances like opioids, alcohol, and nicotine. In one embodiment, for medicinal purposes, the gummy product with THC or CBD can be used for skin conditions (acne and psoriasis). CBD's anti-inflammatory properties have made it a popular ingredient for medicinal purposes in treating acne, psoriasis, and other inflammatory skin conditions. These legal medical uses may vary depending on state and federal cannabis regulations.

FIG. 3C shows, for illustrative purposes only, an example of a gummy product dosage tear-away segment mold profile according to one embodiment. As depicted, the mold is specifically engineered to produce gummy products with thin, strategically weakened sections that function as controlled break points, thereby enabling a user to separate individual segments for consumption with minimal effort. This design addresses a long-standing challenge faced by consumers of traditional solid oral dosage forms, such as compressed medical tablets, which are often difficult to divide accurately into smaller portions. When such tablets are cut, the compressed powder tends to fracture irregularly, resulting in uneven portions where the separated piece may contain substantially less than the prescribed dosage, creating risks of under-or overdosing.

By contrast, the present invention provides a pliable, confectionery medium in which dosage segmentation is pre-engineered into the mold itself. As shown in FIG. 3C, the mold introduces tear lines within the body of the gummy flower, incorporating both the petal portions and the flower head portion into a unified but divisible structure. A narrow section flower head dosage tear-away section 342 connects a petal 300 to the central flower head 320, creating a defined break point for dosage control. Because the gummy material is soft and elastic, the consumer may separate the intended segments without tools or risk of crumbling. FIG. 3C further illustrates a first petal tear-away dosage segment 350 and a second petal tear-away dosage segment 360, each delineated by a tear-away dosage segment line 370. These tear-away dosage segment lines are not random fractures but are deliberately integrated into the recessed flower sections of the silicone mold 380. Importantly, the lines are engineered to provide mechanical weakness for consumer use while remaining non-obstructive to the demolding process, ensuring that the finished gummies can be easily extracted from the mold after cooling without tearing prematurely.

Each of the tear-away dosage segments produced by the mold—including both the first petal segment 350 and the second petal segment 360—contains a predetermined and recommended dosage of the active medicinal ingredient. In addition, the central flower head dosage segment 322 is also designed as a discrete dosage segment, thereby allowing the gummy confection to be divided into multiple consistent portions, each delivering the correct therapeutic amount. In one embodiment, the active ingredients include cannabinoids such as tetrahydrocannabinol (THC) and cannabidiol (CBD). The concentration of THC is carefully regulated, for example, to not more than 0.3 percent on a dry weight basis when derived from hemp, consistent with prevailing legal and regulatory frameworks. This configuration ensures that patients or consumers can reliably obtain uniform doses across different segments of the same gummy product, thereby promoting safety, regulatory compliance, and convenience in administering nutraceutical or pharmaceutical treatments in an edible, consumer-friendly format.

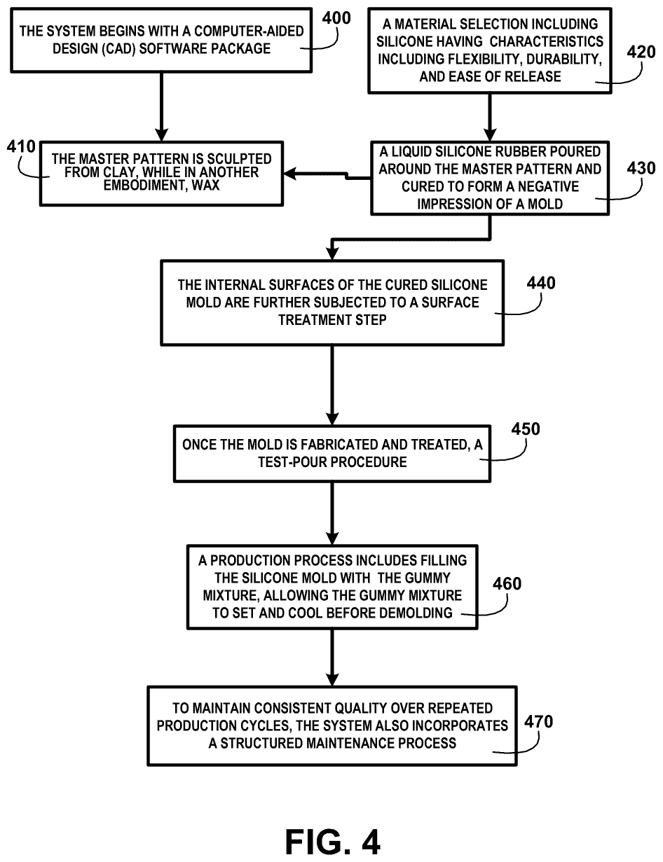

FIG. 4 shows a block diagram providing an overview of a gummy product production system according to one embodiment. As illustrated, the system begins with a computer-aided design (CAD) software package 400, which is used to generate a digital model of the mold based on predetermined specifications, including size, geometric shape, and fine surface details of the gummy product to be produced. These CAD models allow precise control over dosage segmentation features, ornamental textures, and dimensional tolerances, ensuring that the final mold captures both aesthetic and functional requirements. Once the CAD model is completed, a master pattern is fabricated. In one embodiment, the master pattern is sculpted from clay, while in another embodiment, wax 410 is employed to form the initial shape. The master pattern serves as a positive model into which mold-making materials are cast. A material selection step follows, in which silicone is chosen for its advantageous characteristics, including high flexibility, long-term durability, and excellent ease of release 420, all of which are critical for repeated use in confectionery production environments.

After material selection, a liquid silicone rubber compound is prepared and poured around the master pattern, where it is allowed to cure under controlled environmental conditions to form a negative impression of the mold 430, corresponding to the example depicted in FIG. 1. In one embodiment, the internal surfaces of the cured silicone mold are further subjected to a surface treatment step 440. This treatment may include applying a food-grade release agent, a specialized coating, or a micro-textured finish to the mold surface. The purpose of these treatments is to improve the ease of demolding, minimize sticking of the gummy mixture, and extend the operational lifespan of the mold by reducing wear. Once the mold is fabricated and treated, a test-pour procedure 450 is performed in which a trial batch of gummy mixture is deposited into the mold cavities. This test-pour validates that the mold consistently produces the desired gummy shape and dosage features and also establishes operational limits, such as maximum mold temperature, optimum filling techniques, and effective release properties.

Following successful validation, the mold is deployed into the production process, wherein the cavities are filled with the heated gummy mixture, allowed to set, and subsequently cooled under controlled conditions before demolding 460. This ensures that the gummies solidify properly and retain their intended dosage segmentation and surface details. To maintain consistent quality over repeated production cycles, the system also incorporates a structured maintenance process 470. This includes thorough cleaning of the silicone mold between uses to prevent contamination, routine inspections to identify damage or wear, and repair or replacement of molds as needed. Such maintenance ensures that the mold continues to yield gummies of uniform quality and prevents production delays or defective batches. By integrating CAD design, master pattern fabrication, material selection, silicone molding, surface treatment, validation testing, production operations, and maintenance protocols, the system illustrated in FIG. 4 provides a comprehensive and reliable framework for manufacturing precise and reproducible gummy products across multiple production runs.

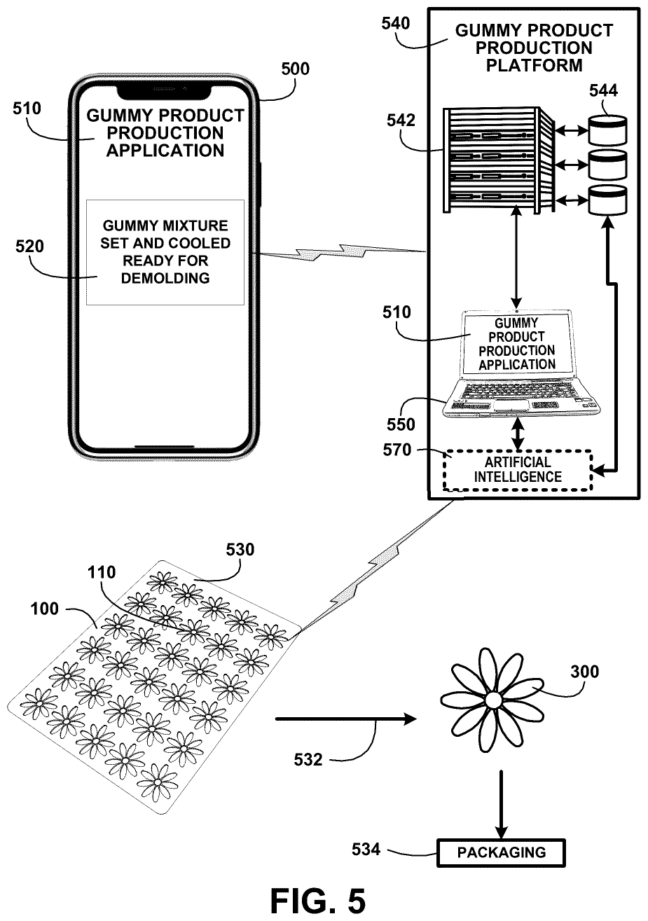

FIG. 5 shows, for illustrative purposes only, an example of a gummy product production platform according to one embodiment. As illustrated, a user's smartphone 500 is configured with a dedicated gummy product production application 510 installed thereon. The application 510 functions as an interface between the user and the automated production system by transmitting dimensional data of the flower-shaped mold to an artificial intelligence (AI) engine 570. Specifically, the application measures or retrieves the recessed design dimensions of the silicone mold 100, including the detailed contours of the recessed features 110, and transmits this data to the AI. The AI 570 then performs volumetric calculations to determine the precise internal volume and corresponding weight of the gummy mixture required to fill each cavity. By aggregating this information across a plurality of molds, the system ensures consistent dosing, efficient material usage, and minimization of waste during production. The production application 510 also provides real-time feedback to operators, including alert messages such as “gummy mixture set and cooled, gummy mixture set and cooled, ready for demolding” 520, thereby guiding the timing of downstream operations and reducing the likelihood of human error.

In one embodiment, the silicone mold 100 is further instrumented with a plurality of sensors 530, each configured to detect and measure process parameters including cavity temperature, ambient humidity, and moisture levels within the gummy products as they cure. These sensors are wirelessly coupled to the centralized gummy product production platform 540, which aggregates and interprets the data. Upon completion of the setting and cooling phases, a demolding process 532 is initiated, enabling the removal of the solidified gummy products from the mold without deformation. Following demolding, each gummy product 300 proceeds to a packaging operation 534, in which the products are prepared for distribution. Throughout the process, the production platform 540 continuously tracks operational progress for each mold in use, while transmitting sensor data to at least one server 542 for further processing. This data is archived within a plurality of databases 544, ensuring traceability, compliance with quality control protocols, and the ability to perform retrospective analysis in case of product or process deviations.

In addition to mobile functionality, a platform computer 550 is also provided and operates the same gummy product production application 510, thereby enabling larger-scale supervisory control and monitoring of the production environment. The computer 550 transmits alerts and notifications to designated personnel, allowing them to monitor system status remotely without being physically present at the production line. Artificial intelligence 570 is further employed by the production platform 540 not only for volumetric analysis but also to optimize operational efficiency. The AI evaluates sensor data, historical production records, and real-time performance metrics to recommend adjustments that may increase throughput, enhance consistency, or reduce waste. In this way, the gummy product production platform provides both automated process management and intelligent decision support, ensuring that high-quality products are consistently produced while maintaining compliance with regulatory and manufacturing standards.

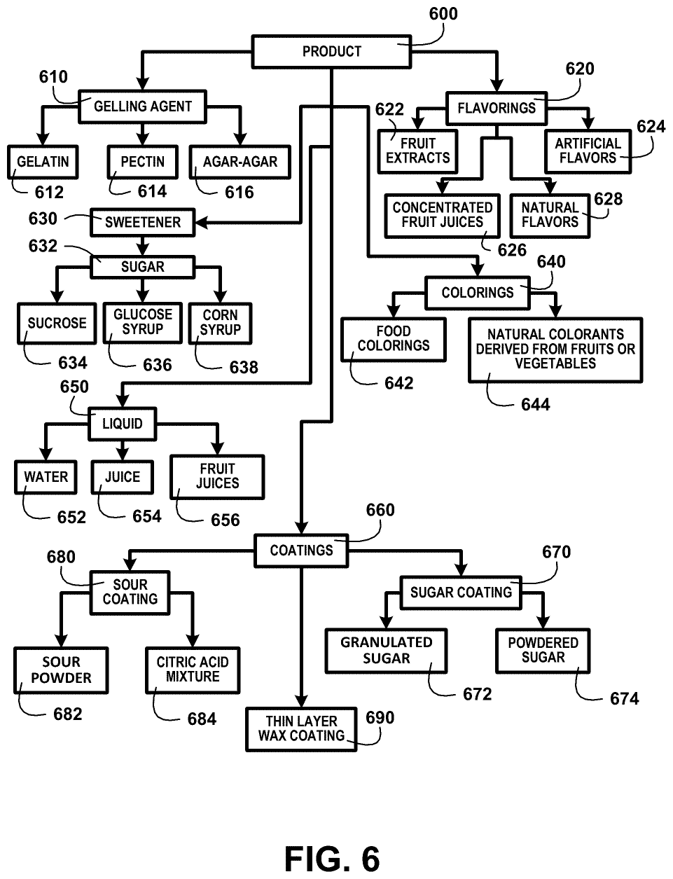

FIG. 6 shows a block diagram providing an overview of gummy product ingredients according to one embodiment. As illustrated, the formulation of the gummy product 600 begins with the selection of an appropriate gelling agent 610, which functions as the structural matrix responsible for giving the gummy its characteristic texture and elasticity. In one embodiment, the gelling agent may be gelatin 612, a protein derived from collagen that provides a chewy and elastic consistency. In another embodiment, pectin 614 is employed, which is a plant-derived polysaccharide commonly extracted from citrus fruits and used in vegan or vegetarian formulations. Alternatively, agar-agar 616, a seaweed-derived gelling material, may be selected for applications requiring higher thermal stability or unique textural attributes. In addition to the gelling agents, flavorings 620 are incorporated to enhance palatability.

These flavorings may include natural fruit extracts 622, laboratory-derived artificial flavors 624, concentrated fruit juices 626, and other natural flavors 628. A sweetener 630 is added to balance taste, which can be selected from sugar 632, sucrose 634, glucose syrup 636, or corn syrup 638, depending on the desired sweetness profile, mouthfeel, and shelf-life characteristics. Furthermore, colorings 640 are integrated into the gummy formulation to provide visual appeal. These may include synthetic food colorings 642 or natural colorants 644 derived from fruits, vegetables, or other botanical sources. Together, these ingredients establish the baseline formulation of the gummy confection prior to the incorporation of medicinal or nutritional additives.

In one embodiment, a liquid component 650 is incorporated into the formulation to dissolve the gelling agent and facilitate mixing of the other ingredients into a uniform solution. The liquid may be selected from water 652, fruit-based juice 654, or blended fruit juices 656, depending on flavor and nutritional requirements. Once the gummy mixture has been prepared and molded, additional exterior coatings 660 may be applied after demolding and cooling.

These coatings serve multiple functions, including enhancement of flavor and mouthfeel, prevention of sticking between individual gummies during storage, and extension of shelf stability by providing a protective barrier. In one embodiment, the coating 660 may include a sugar coating 670, which may be composed of granulated sugar 672, powdered sugar 674, or a combination thereof. In another embodiment, a sour coating 680 is provided, consisting of a sour powder 682 or a citric acid mixture 684, which imparts a tart taste profile favored in certain consumer products. In still another embodiment, the exterior coating may comprise a thin wax coating 690, which provides an additional moisture barrier, enhances handling characteristics, and further prevents sticking of gummies during packaging and transport. Collectively, the ingredients and coatings illustrated in FIG. 6 demonstrate the flexibility of the system to produce gummies with varied flavors, textures, nutritional compositions, and functional properties tailored to specific consumer or medicinal applications.

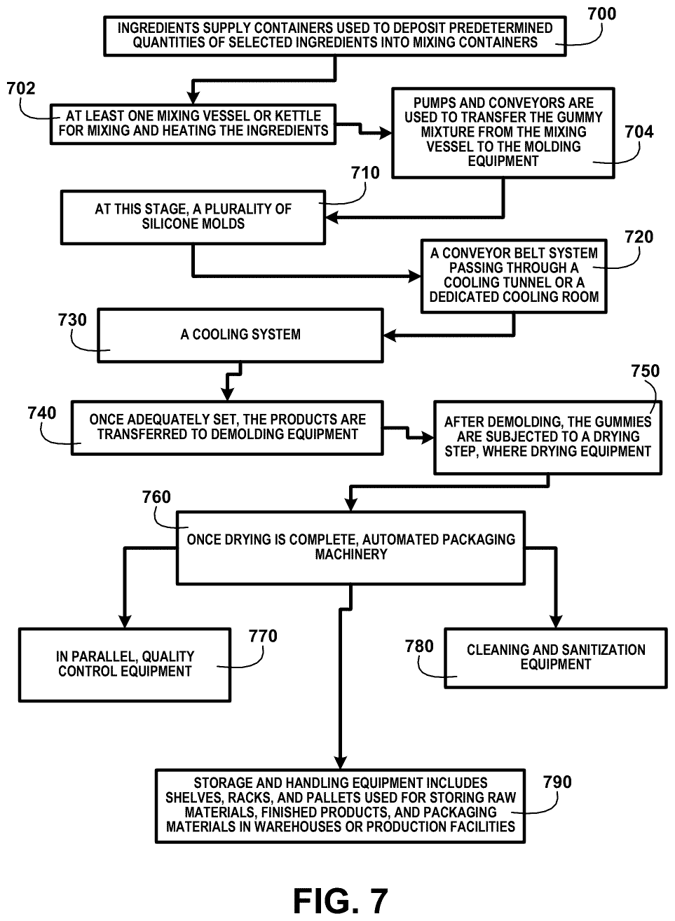

FIG. 7 shows a block diagram providing an overview of gummy product equipment according to one embodiment. As illustrated, the production process begins with ingredients supply containers that are configured to hold and dispense predetermined quantities of raw materials. These materials are deposited into mixing containers 700, where precise metering ensures accurate formulation and consistency across batches. Once dispensed, the ingredients are transferred to at least one mixing vessel or kettle 702, which is designed to both mix and heat the ingredients to form a homogeneous gummy mixture. The vessel or kettle 702 may include temperature control systems, mechanical agitators, and monitoring instruments to ensure that the gelling agents and sweeteners are dissolved and activated at the correct process parameters. Once the mixture has reached the desired consistency, pumps and conveyor systems transfer the heated gummy mixture from the mixing vessel to molding equipment 704. At this stage, a plurality of silicone molds 710 is employed to impart the final form of the gummy products, incorporating both the aesthetic shapes and dosage-segmentation features predetermined by the mold design.

Following deposition into molds, a conveyor belt system 720 transports the filled molds through a controlled cooling environment, such as a cooling tunnel or a dedicated cooling room. This ensures that the gummies are set under stable conditions, preventing premature crystallization or textural inconsistencies. A cooling system 730 may employ forced air, refrigeration, or humidity control to achieve precise cooling rates that stabilize the structure of the gummy mixture. Once adequately set, the products are transferred to demolding equipment 740, which may include vibrating tables or demolding trays that facilitate clean separation of the gummies from the molds without deformation or tearing. After demolding, the gummies are subjected to a drying step, where drying equipment 750, including industrial dehumidifiers or controlled-air systems, removes excess moisture. This step is critical to extending shelf life, preventing microbial growth, and ensuring the desired chewy consistency of the finished product.

Once drying is complete, automated packaging machinery 760 prepares the gummies for distribution. Such machinery may include sealing machines that provide tamper-evident packaging, weighing scales for portion control, and labeling systems that apply regulatory and consumer information. In parallel, quality control equipment 770 is deployed throughout the production line. This equipment may include viscosity meters to monitor mixture consistency, pH meters to maintain chemical balance, moisture analyzers to verify dryness levels, and THC potency analyzers to confirm dosage accuracy in medicinal formulations. These instruments ensure that every batch complies with quality standards and applicable regulations. Cleaning and sanitization equipment 780 is also integrated into the production facility, enabling systematic cleaning of vessels, conveyors, molds, and work surfaces to maintain hygiene and prevent cross-contamination between batches.

Finally, storage and handling equipment 790 is employed to manage both raw materials and finished products. This equipment may include shelving units, storage racks, and pallets, all configured to support proper segregation and organization within warehouses or production facilities. Proper storage ensures that raw ingredients remain uncontaminated, finished gummies retain freshness, and packaging materials are readily available for uninterrupted production cycles. Collectively, the equipment illustrated in FIG. 7 provides an integrated infrastructure that supports the end-to-end manufacturing of gummy products, from raw material handling to packaging and storage, while maintaining rigorous standards for efficiency, safety, and product quality.

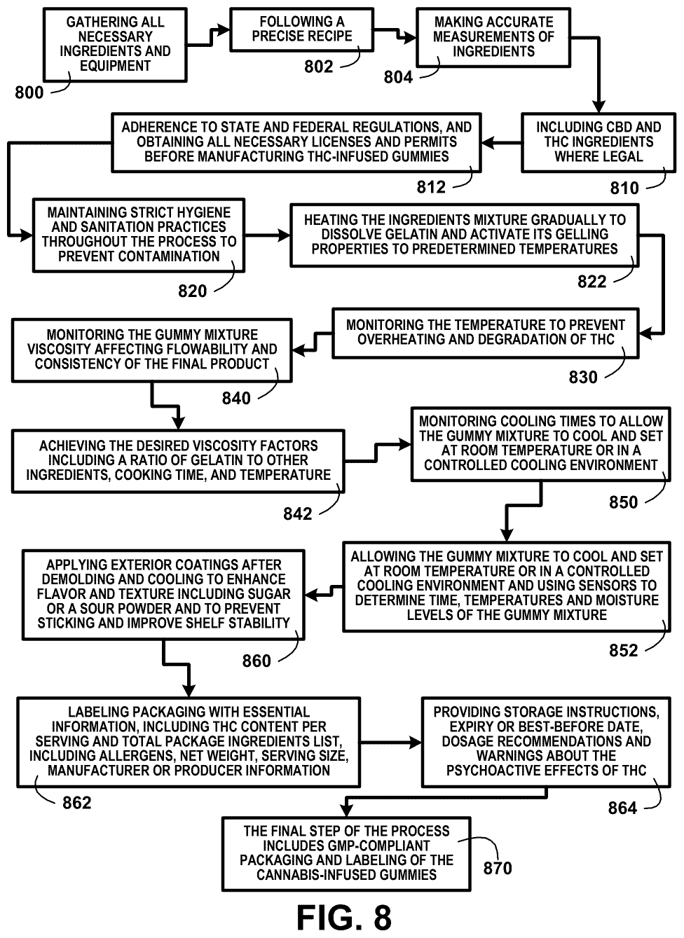

FIG. 8 shows a block diagram of an overview flow chart of a gummy product process according to one embodiment. The process begins with gathering all required ingredients and equipment 800. Production then follows a precise recipe 802, ensuring reproducibility of formulation, and requires accurate measurement of each ingredient 804 to maintain dosage consistency and product uniformity. Where legally permissible, ingredients may include cannabidiol (CBD) and tetrahydrocannabinol (THC) 810. Compliance with applicable laws is mandatory, including adherence to state and federal regulations, and obtaining all necessary licenses and permits before manufacturing THC-infused gummies 812. Throughout the process, strict hygiene and sanitation protocols are observed 820 to prevent contamination. The formulation stage includes heating the mixture of ingredients gradually to dissolve gelatin and activate its gelling properties at predetermined temperatures 822, ensuring a uniform solution and avoiding localized overheating.

Gelling agents form the structural base of a gummy product and are selected to achieve the desired texture. In one embodiment, the gelling agent is pectin, while in another embodiment the gelling agent is gelatin. Both substances thicken liquids and create gel-like textures when boiled, but they are not interchangeable. Gelatin is an animal-derived protein obtained from collagen, while pectin is a plant-derived polysaccharide extracted from citrus fruit peels. Manufacturers often select pectin for vegan or animal-free formulations, whereas gelatin remains common in traditional confectionery.

The functional properties of these gelling agents differ significantly. Gelatin activates at lower temperatures, producing an elastic, chewy texture while remaining flavor-neutral. Pectin requires higher temperatures to gel, stabilizes at room temperature, and enhances flavor release, which can reduce flavoring costs. Pectin is widely used in jams, jellies, and pharmaceutical products and is available in high-methoxyl (HM) and low-methoxyl (LM) varieties, with HM preferred for gummies due to flexibility in processing. Gelatin, beyond confectionery, is also used in capsules, cosmetics, and foods and provides nutritional benefits such as amino acids that support collagen production. When substituting pectin for gelatin, formulation adjustments are required to achieve equivalent texture, and the choice may also be driven by dietary restrictions, since vegetarians and vegans cannot consume gelatin.

Monitoring the temperature to prevent overheating and degradation of THC 830. Temperatures range between 100° C. to 120° C. (212° F. to 248° F.). The steps include monitoring the gummy mixture viscosity, which affects the flowability and consistency of the final product 840. Once the mixture reaches the desired consistency, it is poured into molds. Achieving the desired viscosity factors, including a ratio of gelatin to other ingredients, cooking time, and temperature 842 includes monitoring of all operations. Monitoring cooling times to allow the gummy mixture to cool and set at room temperature or in a controlled cooling environment 850.

Allowing the gummy mixture to cool and set at room temperature or in a controlled cooling environment and using sensors to determine time, temperatures, and moisture levels of the gummy mixture 852. Applying exterior coatings after demolding and cooling to enhance flavor and texture, including sugar or a sour powder, and to prevent sticking and improve shelf stability 860. Labeling packaging with essential information, including THC content per serving for medicinal purposes and total package ingredients list, including allergens, net weight, serving size, manufacturer, or producer information 862. Packaging provides storage instructions, expiry or best-before date, dosage recommendations, and warnings about the psychoactive effects of THC 864.

In one embodiment, the production of the medicinal gummy products described herein is carried out in compliance with Good Manufacturing Practices (GMP) as required by the U.S. Food and Drug Administration (FDA). GMP ensures that each batch of gummies is produced under controlled conditions, with consistent ingredient quality, accurate dosing of medicinal components such as THC or CBD, and traceable records of the production process. Compliance with GMP minimizes risks of contamination, mislabeling, or dosage inconsistency, which is critical when producing gummies intended for medicinal use.

The GMP framework as applied to the present invention encompasses quality management systems, sanitation of mixing and molding equipment, verification of raw materials, and monitoring of personnel handling during production. Specific controls include the use of validated processes for heating, cooling, and demolding, inspection of molds for integrity and cleanliness, and testing of finished gummies for potency and uniformity. Certificates of Analysis (COA) are generated for each batch to confirm that the infused medicinal ingredients meet prescribed dosage levels and safety standards.

In one embodiment, staff involved in production receive compliance training focused on GMP requirements specific to gummy manufacturing, including record-keeping, sanitation protocols, equipment handling, labeling requirements, and adherence to standard operating procedures (SOPs). This training ensures that the gummies produced maintain consistency, safety, and compliance across all production runs.

The final step of the process includes GMP-compliant packaging and labeling of the cannabis-infused gummies 870. Packaging is carried out in tamper-evident, child-resistant containers, which may be individual wrappers or bulk containers depending on the intended distribution channel. Labels identify the active medicinal ingredients, dosage per serving, total cannabinoid content, ingredient lists including allergens, expiration or best-before dates, and required safety warnings, thereby ensuring the gummies are suitable for regulated distribution and retail sale.

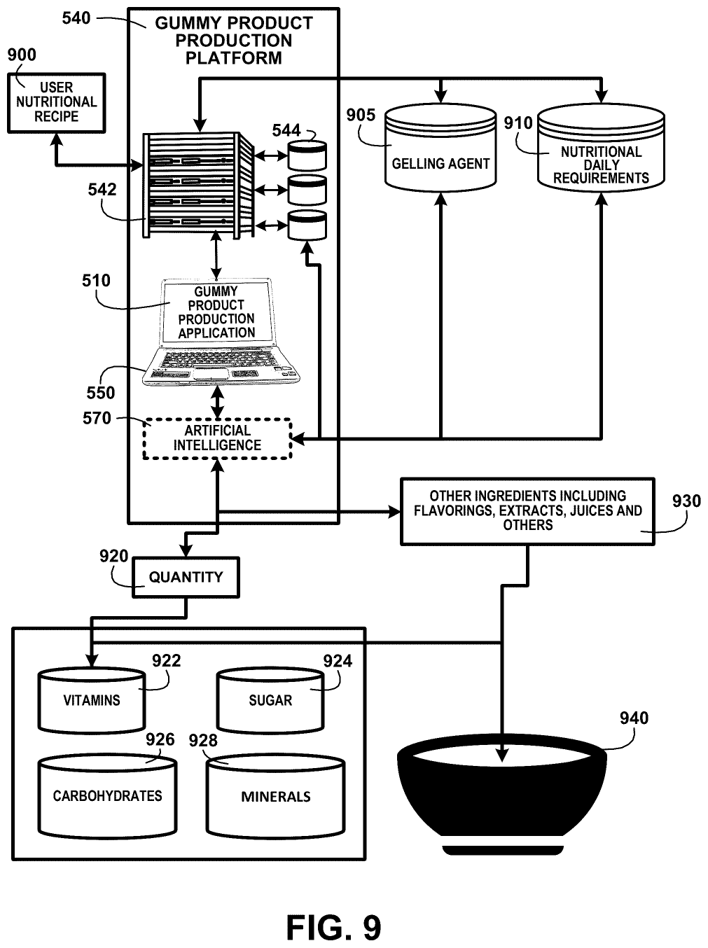

FIG. 9 shows, for illustrative purposes only, an example of an artificial intelligence (AI) nutritional gummy product recipe with daily nutritional requirements adjustment according to one embodiment. As illustrated, the gummy product production platform 540 includes a server 542, a plurality of databases 544, a computer 550, a gummy product production application 510, and an artificial intelligence system 570. These components operate together to process recipe data, calculate nutritional values, and control dispensing and mixing operations. The AI system 570 provides automated analysis of user-specific nutritional requirements and adjusts the formulation of the gummy recipe accordingly, thereby ensuring that the final product delivers precise daily nutrient dosages.

A user nutritional recipe 900 may include a gelling agent 905, nutritional daily requirements 910, and a predetermined quantity 920 of individual ingredient types. These ingredients may include vitamins 922, sugar 924, carbohydrates 926, minerals 928, and other additives such as flavorings, extracts, juices, and other components 930. In one embodiment, the user nutritional recipe 900 data is transmitted to the server 542 of the gummy product production platform 540. The artificial intelligence system 570, coupled to the server 542, records and stores the user's nutritional recipe data within the plurality of databases 544. Once the recipe is validated, the AI system 570 directs a quantity dispensing device to measure and release the predetermined quantities of each ingredient with high accuracy, ensuring compliance with nutritional requirements and regulatory standards.

In one embodiment, the dispensed ingredients are combined manually by the producer in a mixing bowl 940, while in another embodiment, the formulation is scaled to a commercial-grade mixer for larger production volumes. The mixture is agitated until it achieves a predetermined consistency suitable for molding. Once properly prepared, the mixture is deposited into molds configured to define the final gummy shape. The molds may be produced in a variety of forms and sizes, including decorative or functional designs, to accommodate different product lines. In one embodiment, these molds are further adapted to create nutritionally fortified gummies and, where legally permitted, may also be applied to produce THC-infused gummy products for medicinal purposes, allowing the same AI-driven production framework to support multiple categories of gummy formulations.

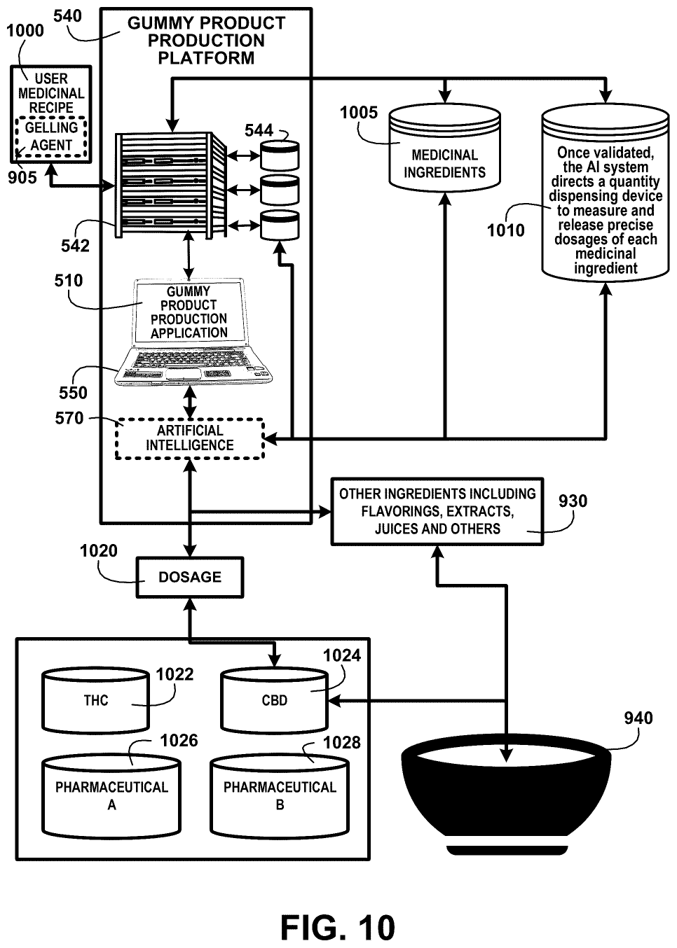

FIG. 10 shows, for illustrative purposes only, an example of an artificial intelligence (AI) medicinal gummy product recipe dosage adjustment according to one embodiment. The system includes a gummy product production platform 540 comprising a server 542, a plurality of databases 544, a computer 550, a gummy product production application 510, and an artificial intelligence system 570. These components are integrated to automate the process of calculating, adjusting, and dispensing medicinal formulations. The AI system 570 is configured to interpret dosage requirements, generate formulation instructions, and control dispensing mechanisms to ensure that each gummy product delivers an accurate therapeutic dose.

A user medicinal recipe 1000 includes a gelling agent 905, as described in FIG. 9, combined with a predetermined dosage 1020 of one or more medicinal ingredients 1005. The medicinal ingredients 1005 may include tetrahydrocannabinol (THC) 1022, cannabidiol (CBD) 1024, pharmaceutical A 1026, pharmaceutical B 1028, and additional excipients such as flavorings, extracts, juices, and other additives 930. In one embodiment, the user medicinal recipe 1000 data is transmitted to the server 542 of the gummy product production platform 540. The artificial intelligence system 570, coupled to the server 542, records and stores the recipe data within the plurality of databases 544. Once validated, the AI system directs a quantity dispensing device to measure and release precise dosages of each medicinal ingredient 1010. This process ensures that the resulting gummy products comply with recommended therapeutic dosages for targeted medical conditions while maintaining batch-to-batch consistency.

In one embodiment, the measured ingredients are combined by the producer in a mixing bowl 940, while in another embodiment, a commercial-grade mixer is employed for larger-scale production. The mixing continues until the ingredients achieve a predetermined uniform consistency suitable for molding. Once prepared, the mixture is poured into molds, which may be designed in a variety of shapes and sizes to meet product specifications. The mold dimensions may be adjusted to account for variations in the concentration of medicinal ingredients, thereby ensuring that each unit provides the correct dosage. In one embodiment, molds are configured to produce THC-infused gummies with precise segmentation and dosage control, ensuring both therapeutic effectiveness and regulatory compliance.

The foregoing has described the principles, embodiments, and modes of operation of the present invention. However, the invention should not be construed as being limited to the particular embodiments discussed. The above-described embodiments should be regarded as illustrative rather than restrictive, and it should be appreciated that variations may be made in those embodiments by workers skilled in the art without departing from the scope of the present invention as defined by the following claims.

What is claimed is:

1. A method of manufacturing gummy confections, comprising:
depositing predetermined quantities of selected ingredients into at least one mixing vessel, the ingredients comprising a gelling agent selected from the group consisting of gelatin, pectin, and agar-agar, a sweetener selected from the group consisting of sucrose, glucose syrup, and corn syrup, a liquid carrier comprising at least one of water or fruit juice, at least one flavoring component, and at least one cannabinoid-containing ingredient;
heating the deposited ingredients within the mixing vessel to a predetermined temperature range sufficient to dissolve the gelling agent and activate its gelling properties while maintaining controlled thermal conditions that prevent degradation of the cannabinoid-containing ingredient;
agitating the heated mixture until a substantially homogeneous gummy confection mixture is formed with a uniform distribution of the cannabinoid-containing ingredient;
transferring the homogeneous gummy confection mixture into a molding assembly comprising a plurality of cavities, each cavity being shaped to produce a flower-shaped gummy confection including a central portion corresponding to a flower head and a plurality of radially extending petal portions joined to the central portion by narrow tear-away sections;
forming within the molding assembly predetermined dosage segmentation lines configured as reduced-thickness tear-away sections that mechanically weaken junctions between each petal portion and the central portion such that the resulting gummy confection permits a user to manually separate individual petal portions from the flower head without tools;
predetermining dosage levels such that each petal portion and the central portion of the flower-shaped gummy confection contains a controlled amount of the cannabinoid-containing ingredient based on volumetric displacement of the corresponding cavity region of the mold and the concentration of cannabinoid in the homogeneous mixture;
cooling the molding assembly under controlled conditions until the gummy confection mixture solidifies within the cavities while preserving the predetermined dosage segmentation lines;
demolding the solidified flower-shaped gummy confections from the cavities of the molding assembly while maintaining integrity of the tear-away segmentation lines; and
applying at least one exterior coating to the demolded flower-shaped gummy confections, the coating comprising at least one of sugar, edible wax, or powdered fruit, the coating being configured to reduce surface tackiness, enhance flavor, and improve storage stability without interfering with tear-away functionality of the petal portions.

2. The method of claim 1, wherein heating the gummy confection mixture is performed within a predetermined temperature range between 100° C. and 120° C. (212° F. to 248° F.) to dissolve the gelling agent and preserve the chemical stability of the cannabinoid-containing ingredient.

3. The method of claim 1, wherein applying the exterior coating comprises depositing a sugar coating including granulated sugar and powdered sugar onto the surface of the gummy confections to improve palatability and reduce surface tackiness.

4. The method of claim 1, wherein mixing further comprises incorporating a liquid ingredient selected from the group consisting of water, fruit juice, and blended fruit juices to dissolve the gelling agent and promote uniform mixing of the ingredients.

5. The method of claim 1, wherein the gelling agent ingredient controls a texture, thermal properties, and dietary requirements of the gummy confection.

6. The method of claim 1, wherein the flavoring ingredient is configured to enhance the sensory characteristics of the gummy confection.

7. The method of claim 1, wherein each petal portion and the central portion is dimensioned to contain a predetermined dosage amount of the cannabinoid-containing ingredient, thereby enabling a user to separate and consume individual dosage portions without cutting or measuring.

8. The method of claim 1, wherein the molding assembly is configured such that the volume of each petal portion is calculated relative to the concentration of the cannabinoid-containing ingredient in the homogeneous mixture, and wherein the volume of each petal portion is dimensioned to correspond to a unit dosage to comply with regulatory limits, thereby ensuring consistent dosage uniformity across production batches.

9. A method of manufacturing gummy confections, comprising:
depositing predetermined quantities of selected ingredients into at least one mixing vessel, the ingredients comprising a gelling agent selected from the group consisting of gelatin, pectin, and agar-agar, a sweetener selected from the group consisting of sucrose, glucose syrup, and corn syrup, a liquid carrier comprising at least one of water or fruit juice, at least one flavoring component, and at least one cannabinoid-containing ingredient;
heating the deposited ingredients within the mixing vessel to a predetermined temperature range sufficient to dissolve the gelling agent and activate its gelling properties while maintaining controlled thermal conditions that prevent degradation of the cannabinoid-containing ingredient;
agitating the heated mixture until a substantially homogeneous gummy confection mixture is formed with a uniform distribution of the cannabinoid-containing ingredient;
transferring the homogeneous gummy confection mixture into a molding assembly comprising a plurality of cavities, each cavity being shaped to produce a flower-shaped gummy confection including a central portion corresponding to a flower head and a plurality of radially extending petal portions joined to the central portion by narrow tear-away sections;
forming within the molding assembly predetermined dosage segmentation lines configured as reduced-thickness tear-away sections that mechanically weaken junctions between each petal portion and the central portion such that the resulting gummy confection permits a user to manually separate individual petal portions from the flower head without tools;
wherein the molding assembly is configured such that the volume of each petal portion is calculated relative to the concentration of the cannabinoid-containing ingredient in the homogeneous mixture, and wherein the volume of each petal portion is dimensioned to correspond to a predetermined unit dosage to comply with regulatory limits, thereby ensuring consistent dosage uniformity across production batches;
predetermining dosage levels such that each petal portion and the central portion of the flower-shaped gummy confection contains a controlled amount of the cannabinoid-containing ingredient based on volumetric displacement of the corresponding cavity region of the mold and the concentration of cannabinoid in the homogeneous mixture;
cooling the molding assembly under controlled conditions until the gummy confection mixture solidifies within the cavities while preserving the predetermined dosage segmentation lines; and
demolding the solidified flower-shaped gummy confections from the cavities of the molding assembly while maintaining integrity of the tear-away segmentation lines.

10. The method of claim 9, wherein heating the gummy confection mixture is performed within a predetermined temperature range between 100° C. and 120° C. (212° F. to 248° F.) to dissolve the gelling agent and preserve the chemical stability of the cannabinoid-containing ingredient.

11. The method of claim 9, wherein applying the exterior coating comprises depositing a sugar coating including granulated sugar and powdered sugar onto the surface of the gummy confections to improve palatability and reduce surface tackiness.

12. The method of claim 9, wherein mixing further comprises incorporating a liquid ingredient selected from the group consisting of water, fruit juice, and blended fruit juices to dissolve the gelling agent and promote uniform mixing of the ingredients.

13. The method of claim 9, wherein the gelling agent ingredient controls a texture, thermal properties, and dietary requirements of the gummy confection.

14. The method of claim 9, wherein the flavoring ingredient is configured to enhance the sensory characteristics of the gummy confection.

15. The method of claim 9, wherein each petal portion and the central portion is dimensioned to contain a predetermined dosage amount of the cannabinoid-containing ingredient, thereby enabling a user to separate and consume individual dosage portions without cutting or measuring.

16. A method of manufacturing gummy confections, comprising:
depositing predetermined quantities of selected ingredients into at least one mixing vessel, the ingredients comprising a gelling agent selected from the group consisting of gelatin, pectin, and agar-agar, a sweetener selected from the group consisting of sucrose, glucose syrup, and corn syrup, a liquid carrier comprising at least one of water or fruit juice, at least one flavoring component, and at least one cannabinoid-containing ingredient;
wherein mixing further comprises incorporating a liquid ingredient selected from the group consisting of water, fruit juice, and blended fruit juices to dissolve the gelling agent and promote uniform mixing of the ingredients;
heating the deposited ingredients within the mixing vessel to a predetermined temperature range sufficient to dissolve the gelling agent and activate its gelling properties while maintaining controlled thermal conditions that prevent degradation of the cannabinoid-containing ingredient;
agitating the heated mixture until a substantially homogeneous gummy confection mixture is formed with a uniform distribution of the cannabinoid-containing ingredient;
transferring the homogeneous gummy confection mixture into a molding assembly comprising a plurality of cavities, each cavity being shaped to produce a flower-shaped gummy confection including a central portion corresponding to a flower head and a plurality of radially extending petal portions joined to the central portion by narrow tear-away sections;
wherein each petal portion and the central portion recesses are dimensioned to contain a predetermined dosage amount of the cannabinoid-containing ingredient, thereby enabling a user to separate and consume individual dosage portions without cutting or measuring;
forming within the molding assembly predetermined dosage segmentation lines configured as reduced-thickness tear-away sections that mechanically weaken junctions between each petal portion and the central portion such that the resulting gummy confection permits a user to manually separate individual petal portions from the flower head without tools;
wherein the molding assembly is configured such that the volume of each petal portion is calculated relative to the concentration of the cannabinoid-containing ingredient in the homogeneous mixture, and wherein the volume of each petal portion is dimensioned to correspond to a unit dosage to comply with regulatory limits, thereby ensuring consistent dosage uniformity across production batches;
predetermining dosage levels such that each petal portion and the central portion of the flower-shaped gummy confection contains a controlled amount of the cannabinoid-containing ingredient based on volumetric displacement of the corresponding cavity region of the mold and the concentration of cannabinoid in the homogeneous mixture;
cooling the molding assembly under controlled conditions until the gummy confection mixture solidifies within the cavities while preserving the predetermined dosage segmentation lines;
demolding the solidified flower-shaped gummy confections from the cavities of the molding assembly while maintaining integrity of the tear-away segmentation lines; and
applying at least one exterior coating to the demolded flower-shaped gummy confections, the coating comprising at least one of sugar, edible wax, or powdered fruit, the coating being configured to reduce surface tackiness, enhance flavor, and improve storage stability without interfering with tear-away functionality of the petal portions.

17. The method of claim 16, wherein heating the gummy confection mixture is performed within a predetermined temperature range between 100° C. and 120° C. (212° F. to 248° F.) to dissolve the gelling agent and preserve the chemical stability of the cannabinoid-containing ingredient.

18. The method of claim 16, wherein applying the exterior coating comprises depositing a sugar coating including granulated sugar and powdered sugar onto the surface of the gummy confections to improve palatability and reduce surface tackiness.

19. The method of claim 16, wherein mixing further comprises incorporating a liquid ingredient selected from the group consisting of water, fruit juice, and blended fruit juices to dissolve the gelling agent and promote uniform mixing of the ingredients.

20. The method of claim 16, wherein the gelling agent ingredient controls a texture, thermal properties, and dietary requirements of the gummy confection.
